(19) United States
(12) Patent Application Publication
(10) Pub. No.: US 2022/0326761 A1
(43) Pub. Date: Oct. 13, 2022

(54) VIRTUAL REALITY INTERACTIVE SYSTEM FOR NEURO-MEDITATION AND NEURO-CONCENTRATION TRAINING

(71) Applicant: NEUROGRESS LIMITED, Hong Kong (HK)

(72) Inventors: Iuliia Olegovna LOBODA, g. Tomsk (RU); Konstantin Yur'evich GORBUNOV, g. Novosibirsk (RU)

(21) Appl. No.: 17/640,517

(22) PCT Filed: Sep. 5, 2019
(86) PCT No.: PCT/RU2019/000620

§ 371 Date: Mar. 4, 2022

(51) Int. Cl. G06F 3/01; A61B 5/16; A61B 5/291; A61B 5/375; A61B 5/00; G16H 20/70; A61M 21/02; G02B 27/00; G02B 27/01

(52) U.S. Cl. CPC G06F 3/011; A61B 5/165; A61B 5/291; A61B 5/375; A61B 5/6803; G16H 20/70; A61M 21/02; G02B 27/0093; G02B 27/0179; G02B 27/017; A61M 2021/0027

(57) ABSTRACT

A virtual reality (VR)-based interactive system for a neuro-meditation and neuro-concentration training. The system has a VR device with a display. The display is configured to provide a VR content. The system also includes a neural interface apparatus having a plurality of electrodes, each electrode is configured to receive a bio-signal data from a user, and a computing device having a processor. The computing device communicates with the neural interface apparatus and the VR device. The computing device also continuously receives the bio-signal data from the electrodes. The processor determines a feedback based on the bio-signal data and predetermined values and communicates the feedback to the VR device, the feedback being displayed as the VR content on the display of the VR device.

### BACKGROUND

The present invention generally relates to virtual reality (VR)-based interactive systems. More specifically, the present invention relates to a VR-based interactive system for neuro-meditation and neuro-concentration training.

A focus style neuro-meditation employs the technique of holding attention on a single object. This technique is shown to increase activation of the frontal lobes of a human brain and help train the mind to improve a variety of cognitive functions including sustaining attention, reducing mind wandering, improving reaction time, and working memory.

A VR environment may be displayed on the display to provide a computer simulation of real world elements. Such immersive VR environment can aid and increase activation of the brain's functions and improve cognitive interactions. As the result of the activation of the brain functions by the VR environment, bio-signals can be generated that are then can be measured, monitored and quantified using an electroencephalogram (“EEG”). Generally, an EEG will measure brainwaves in an analog form. Then, these brainwaves may be analyzed either in their original analog form or in a digital form after an analog to digital conversion.

Further, brain computer interfaces (BCIs) have been developed that allow users to communicate with and control devices using brainwave signals that are measured by EGG.

### SUMMARY

Generally, a neuro-meditation is a conscious gradual process directed at psychological and physical relaxation. A neuro-concentration process is directed at focusing one's attention. Similarly, a focus style neuro-meditation employs the technique of holding attention on a single object. The focus style neuro-meditation can combine both processes, the neuro-meditation and the neuro-concentration to increase activation of the frontal lobes of a human brain and help train the mind to improve a variety of cognitive functions including sustaining attention, reducing mind wandering, improving reaction time, and working memory.

Both the neuro-meditation and the neuro-concentration can be implemented by using images. In fact, a virtual reality (VR) environment can be employed in both processes to display an image on a display of a VR device, for example, a VR helmet, to provide a computer simulation of real world elements. Such immersive VR environment can aid and increase activation of the brain's functions and improve cognitive interactions, thereby improving neuro-meditation and the neuro-concentration processes.

As the result of the activation of the brain functions by the images displayed via the VR environment in the neuro-meditation and the neuro-concentration processes, bio-signals can be generated that are then can be measured, monitored and quantified using an electroencephalogram (“EEG”). The EEG will measure brainwaves in an analog form. Then, these brainwaves may be analyzed either in their original analog form or in a digital form after an analog to digital conversion by brain computer interfaces (BCIs). The BCIs can then provide a neuro-feedback in the form of, for example, a visual content displayed in the VR environment to communicate then current user's state.

Known solutions for VR-based neuro-meditation and/or neuro-concentration techniques lack means for real-time tracking and updating of a user's data that is transmitted from the bio-sensors placed on the user, which is measured by an EEG. In particular, these neural interfaces (i.e., brain computer interfaces (BCIs)) may provide for neuro-meditation and neuro-concentration quantitative coefficients when determining the user's state, but they do not implement and consider a stability component and parameters in their determination. The determination of the stability of the user's state on every stage of the neuro-meditation and neuro-concentration process allows the process to achieve vastly superior results. The values of neuro-meditation and neuro-concentration quantitative can vary significantly several times per second. It is generally assumed that steady high values of meditation and concentration coefficients correspond to the user approaching or achieving the corresponding to such coefficient state, relaxation or concentration, respectively. However, merely achieving the desired level of relaxation (high meditation coefficient) or level of focus (high concentration coefficient) is not sufficient to provide for the effective and long-lasting results of the neuro-meditation and neuro-concentration processes. In order to achieve the elevated state of relaxation and focus for prolonged periods of time, the process of achieving the state must accurately and gradually determine the stability of the then current user' state during all stages of the process. That is, it is desirable to determine whether the user has reached or failed to reach the desired intermediate state in a specific time period during neuro-meditation and/or neuro-concentration processes. The intermediate determination of the user's state can be measured, for example, every ten second or less. This allows gradual changes in meditation and/or concentration processes to optimize the processes and to achieve stable and enduring results for the user.

Thus, the object of the present invention is a VR-based interactive system for neuro-meditation and neuro-concentration training that not only records the achievement of a given meditation and concentration coefficients values, but rather analyzes the coefficients' stability or instability in a given time period during neuro-meditation and neuro-concentration processes. This in turn allows gradual changes, adjustments and optimization of the meditation or concentration training processes, and thereby achieving sustainable results in improving and enhancing users' psychological state, ability to maintain focus, decision-making capabilities and overall cognitive abilities. Moreover, embodiments of the present invention have shown to mitigate psychological stress in users. In particular, a virtual reality (VR)-based interactive system of the present invention and related methods have shown to significantly reduce the psychological stress levels associated with competitive occupational and academic environments and improving users' productivity, focus and cognitive abilities.

In view of the problems and drawbacks described above, in one aspect, the present invention provides the virtual reality (VR)-based interactive system for a neuro-meditation and neuro-concentration training. The system includes a VR device with a display. The display is configured to provide a VR content. The system also includes a neural interface apparatus having a plurality of electrodes, each electrode is configured to receive a bio-signal data from a user, and a computing device having a processor. The computing device communicates with the neural interface apparatus and the VR device. The computing device also continuously receives the bio-signal data from the electrodes. The processor determines a feedback based on the bio-signal data and predetermined values and communicates the feedback to the VR device, the feedback being displayed as the VR content on the display of the VR device.

In another aspect, the present invention provides a method for the neuro-meditation and the neuro-concentration training. The method includes providing a VR device with a display. The display can be configured to provide a VR content. The method includes determining a bio-signal data of a user by a neural interface apparatus having a plurality of electrodes, each electrode is configured to receive the bio-signal data. The method further includes providing a computing device having a processor. The computing device communicates with the neural interface apparatus and the VR device and continuously receives the bio-signal data from the electrodes. The method also provides for determining by the processor a feedback based on the bio-signal data and predetermined values and communicating the feedback to the VR device, displaying the feedback as the VR content on the display of the VR device.

### BRIEF DESCRIPTION OF THE DRAWINGS

In order for the invention to be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, aspects of the invention will be described and explained with additional specificity and detail through the use of the accompanying drawings.

FIG. 1 depicts a front view of a VR-based interactive system according to embodiments of the invention;

FIG. 2 depicts a side view of the VR-based interactive system in accordance with embodiments of the invention;

FIG. 3 depicts the VR-based interactive system that further includes two single-hand controllers in accordance to embodiments of the invention;

FIG. 4 depicts the VR-based interactive system according to embodiments of the invention; and

FIG. 5 depicts a diagram illustrating a method for the neuro-meditation and the neuro-concentration training according to embodiments of the invention.

### DETAILED DESCRIPTION

Reference to “a specific embodiment” or a similar expression in the specification means that specific features, structures, or characteristics described in the specific embodiments are included in at least one specific embodiment of the present invention. Hence, the wording “in a specific embodiment” or a similar expression in this specification does not necessarily refer to the same specific embodiment.

Hereinafter, various embodiments of the present invention will be described in more detail with reference to the accompanying drawings. Nevertheless, it should be understood that the present invention could be modified by those skilled in the art in accordance with the following description to achieve the excellent results of the present invention. Therefore, the following description shall be considered as a pervasive and explanatory description related to the present invention for those skilled in the art, not intended to limit the claims of the present invention.

Reference to “an embodiment,” “a certain embodiment” or a similar expression in the specification means that related features, structures, or characteristics described in the embodiment are included in at least one embodiment of the present invention. Hence, the wording “in an embodiment,” “in a certain embodiment” or a similar expression in this specification does not necessarily refer to the same specific embodiment.

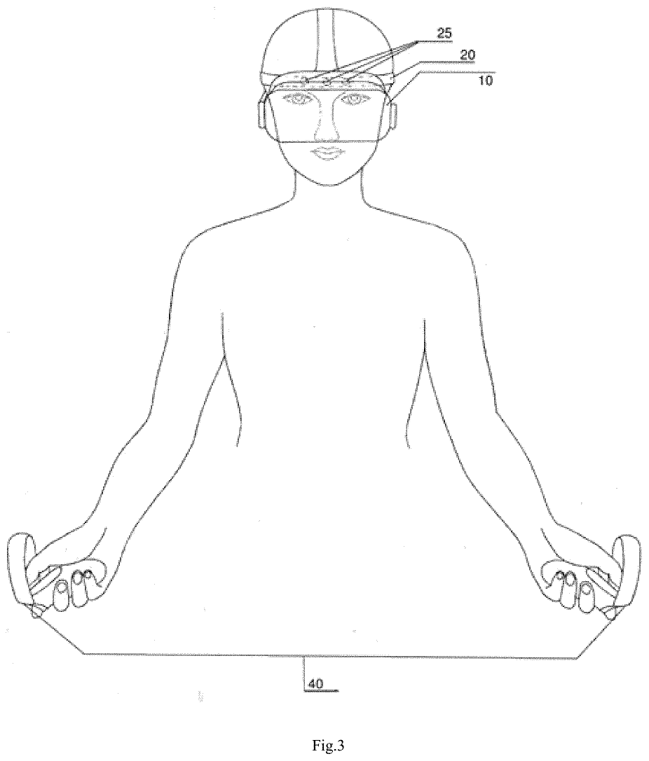

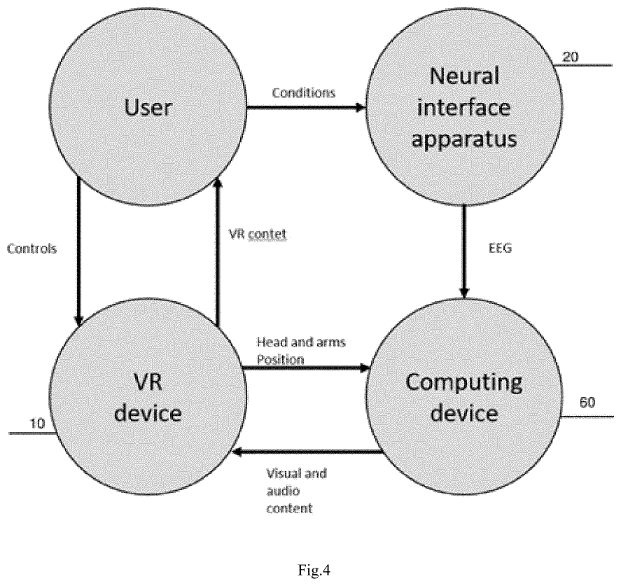

In view of the foregoing and other exemplary problems, drawbacks, and disadvantages of the conventional methods and systems, a VR-based interactive system 100 for neuro-meditation and neuro-concentration training is provided that includes a VR device 10, a neural interface apparatus 20 with a plurality of the electrodes 25, a computing device 60 (shown on FIG. 4), and single-hand controllers 40 (shown on FIG. 3) for interacting with a VR environment. The VR device 10 can include a display (not shown) configured to display three-dimensional (3D) images.

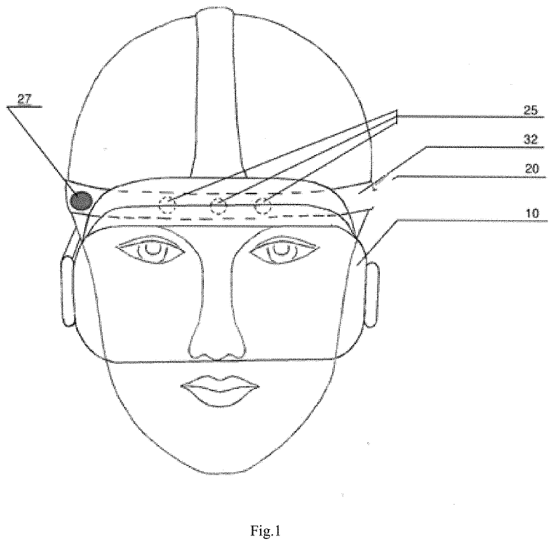

In FIG. 1, the front view of the VR-based interactive system 100 is illustrated with the neural interface apparatus 20 and the VR device 10. The VR device 10, for example, can include one or more speakers, microphones, and/or head phones. The neural interface apparatus 20 includes the electrodes 25 positioned, for example, in the frontal lobe section of user's head as shown in FIGS. 1-3. The electrodes 25 can include a brainwave sensor that is configured to receive a bio-signal data from the user, and the received bio-signal data in turn can include at least brainwave data of the user. The electrodes 25 can measure, for example, electrical bio-signals such as EEG, Electromyograph (EMG), and Electrooculography (EOG).

In addition, the neural interface apparatus 20 can include a positioning sensor 27 for measuring movement of the neural interface apparatus 20. The positioning sensor 27 detects the three-dimensional coordinates of the neural interface apparatus 20 and thereby can detect user's orientation or movement of the VR device 10 and the VR environment. The positioning sensor 27, for example, can include one or more accelerometers and/or gyroscopes. The neural interface apparatus 20 can have, for example, Baud rate of 9600, three frontal electrodes ((EEG), a ground electrode (GND), and a reference electrode (REF)), a Bluetooth 2.0 with a radius of 10 meters, and a battery capable of operational time of one hour. The VR device 10 can have an mage refresh rate of 60 Hz, display resolution of 1920×1080 (or 960×1080 for each eye), and viewing angle of 80°.

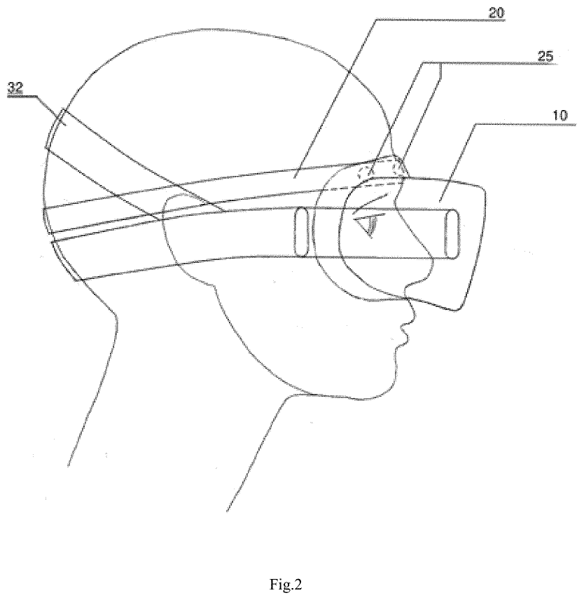

FIG. 2 illustrates a side view of the system 100 having, for example, the neural interface apparatus 20 and the VR device 10. The neural interface apparatus 20 and the VR device 10 can be positioned on the user's head so that the user views the VR environment as being directly in front of the user's eyes even when the user's head is tilted. The VR device also can include straps 32. Additional bio-signal sensors may be positioned along one or more of the straps 32 to sense brainwave activity of the user through the user's head. The straps 32 can include other devices such as touch input devices and the like.

FIG. 3 illustrates the system 100 which further includes two single-hand controllers 40 for interacting with the VR environment. The controllers 40 can be motion controllers to provide motion tracking of user's hands and relate the user's motions to the VR device 10. The controllers 40 can also include analog sticks, face buttons and triggers to assist the user in communications with the VR environment.

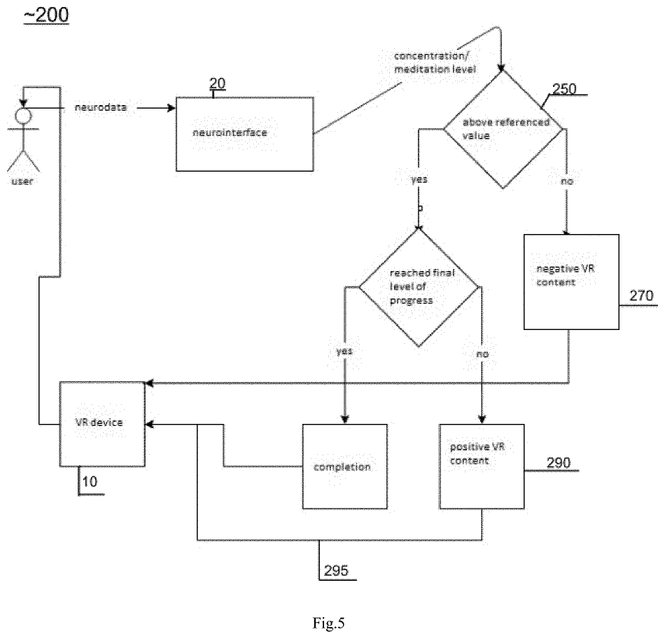

As shown in FIG. 4, the system 100 also includes the computing device 60. The computing device 60 can send and receive data as well as other communication from the neural interface apparatus 20, including the controllers 40, and the VR device 10. This content allows the creation of the VR environment. The VR environment provides a computer simulation of physical elements, a VR content 270, 290 (further described in FIG. 5). The user may interact with the VR environment using various input devices including bio-signal sensors (shown in FIGS. 1-3) and in response, the VR content may be modified to provide the neuro-feedback to the user (as shown in FIG. 4).

The computing device 60 can be connected to the neural interface apparatus 20 and the VR device 10 over the Internet, Bluetooth® network or any other suitable means. Alternatively, the computing device 60 can be incorporated into the neural interface apparatus 20 and/or the VR device 10.

The computing device 60 can include additional commuting devices, each carrying out their respective functions and processing data.

As shown in FIG. 4, the user by way of the controllers 40 or other known means can control the VR device 10, while the VR device 10 communicates visual images (the VR content 270) of the VR environment to the user in real-time. The VR device 10 and controllers 40 can communicate data, for example, data that includes the user's head and hand position to the computing device 60. The neural interface apparatus 20 can communicate to the computing device 60, for example, the bio-signal data that includes at least brainwave data of the user (as shown in FIG. 4). That is, a processor (not shown), provided on the computing device 60, receives the brainwave data (e.g., EEG of the frontal brain lobes) from the neural interface apparatus 20.

According to embodiments of the present invention, a method 200 for the neuro-meditation and the neuro-training, as illustrated in FIG. 5, is provided. The method 200 can include two step approach, a meditation stage (illustrated in FIG. 5) and a concentration stage (not shown). According to embodiments of the present invention, the meditation stage is performed before the concentrations step. For the meditations step, a meditation coefficient can be used. The meditation coefficient can be a standardized value that characterizes the level of relaxation of the user. For the concentration stage, a concentration coefficient can be used. The concentration coefficient can be a standardized value that characterizers the level of concentration of the user.

In order to perform the method 200, the brainwave data collected by the neural interface apparatus 20 (via electrode array 25) is continuously transmitted to the computing device 60, where it is analyzed and evaluated by the processor against the applicable coefficient, for example, in the case of the mediation step, the meditation coefficient. In accordance with the results of the analysis, a command (feedback) from the computing device is sent to the VR device 10 to create particular images, in particular, the VR content 270, 290 in the VR environment. The VR content 270, 290 changes with continuous evaluation of the brainwave data received by the electrodes 25.

According to embodiments of the invention, the method 200 can include six levels. The levels 1-3 are associated with the mediations steps, while the levels 4-6 are associated with the concentration levels. For each level, a referenced value 250 of the meditation coefficient or the concentration coefficient is determined, respectively. The reference value 250 must be achieved and sustained by the user in order to move on to the next level of the meditation levels or concentration levels. Each level 1-6 can further include a number of sublevels, for example, a first sublevel of meditation (Forest as shown on Table 1 below) can include up to seven sublevels. At each sublevel, a new stimulus (the VR content) is presented to the user. For each sublevel, a positive and negative dynamic of the VR content 270, 290 are provided (as illustrated in Table 1). A positive dynamic 290 of the VR content 290 leads to a completion 295 of the sublevel, after which the next sublevel begins or, if it was the last sublevel that has been completed, the next level. A negative dynamic of the VR content 270 can terminate in the beginning of the current sublevel. In other words, the negative dynamic of the VR content 270 (the user is performing poorly) cannot lead to the beginning of the level or the training exercise.

According to embodiment of the present invention, the processor determines an actual value of the user's state from the brainwave data collected by the neural interface apparatus 20 every second. The processor determines a meditation actual value and a concentration actual value. The meditation actual value or the concentration actual value is compared to the respective reference value 250 (the meditation reference value or concentration reference value) for then current level. If the meditation actual value or the concentration actual value is greater than the applicable reference value 250, then the content changes in a positive direction, for example an image of the VR content 290 gets larger or a new image appears. To the contrary, if the meditation actual value or the concentration actual value is lower than the applicable reference value 250, then the VR content 270 changes negatively, for example, an image of the VR content 270 gets smaller or disappears. The actual values are often unstable. For example, the meditation actual value can be 0.7, and within a second period, changes to 0.2. However, if the user's state is stable, the actual values usually fall in a certain range. If the actual values are predominantly higher than the reference values 250, then the user completes the sublevel and the level, respectively. The negative dynamic known to be short and can be not noticed by the user. If the user cannot reach the desired state, then the VR content 270 changes predominantly in the negative direction. As a result, the user remains at the current sublevel until the user can achieve a state of meditation or concentration, which then will result in consistently greater actual values. As a result, the method not only records the achievement of the actual values, but also considers their stability or instability.

Each sublevel has a minimum period during which the sublevel has to be completed. The minimum time period allows for a demonstration of the VR content 270, 290 without resulting in the negative dynamic. In other words, even if the user has quickly reached the desired state, to complete the sublevel the user will have to pass through the entire sublevel. The total time necessary to complete all levels depends on the initial state and the level of preparation of the user. The user can start the program from any level. However, if the desired meditation and concentration values are not reached, the user cannot complete the level or sublevel.

The reference values 250, the minimum time necessary to complete the levels and sublevels, and content dynamic for each level according to embodiments of the present invention are set forth in the Table 1. Various complexities for the levels are possible. In other words, for the less challenging levels (initial levels, for example, level 1, sublevel 1), the reference values 250 can be lower, and at the more difficult levels the reference values 250 can be higher. That is, untrained users will be able to first learn to develop and maintain a less stable state, and then move on to more difficult tasks. Content at different levels will remain the same.

The achievement of desired state of meditation and concentration is the user's goal. The purpose of the VR content 270, 290 presented through the VR device 10 is to demonstrate the progress in the process of meditation or concentration. According to embodiments of the present invention, each level has a minimum time to achieve the desired state of meditation or concentration, for example 2-2.5 minutes.

According to embodiments of the present invention, the levels are set forth in the Table 1 and are described below.

A level “forest” consists of five sublevels. At the beginning of each sublevel, a visual image is presented to the user, i.e., a statue of an animal. As the user enters the relaxation state the statue of animal is reduced in size and can disappear. Subsequently, the next sublevel begins. At each sublevel, the process of reducing the statue is repeated with a different animal. After passing all the sublevels of level one, the user moves on to the next level 2.

A level “Japanese garden” consists of seven sublevels. At each sublevel, a new stone “grows” out of the ground. Stone size increases as the user's relaxation state improves. At the end of each sublevel, the stone takes a given place, after which a transition to the next sublevel takes place.

A level “island” consists of nine sublevels. During each sublevel, the daytime changes (e.g., the view of the sky and the level of light change) from sunrise to sunset, which is the main indicator of relaxation. At sublevels 1-3, the user is “positioned” on the ship going to the island. The end of the sublevel is additionally marked by the movement of the ship relative to the island. In sublevels 4-9, the user “moves” around the island. After passing through all the sublevels of level 3, the meditation stage ends, and the user advances to the concentration stage.

A “target” level consists of five sublevels. At each sublevel, the user has a task of concentrating on a target at which the archer is shooting. Successful shot means that the user has reached the desired level of concentration, and consequently the end of each respective sublevel.

A level “tower” consists of seven sublevels. At each sublevel, the user's task is to concentrate on moving the three rings of the “Tower of Hanoi”. As the user concentrates, the ring gradually disappears from their current position and simultaneously appears on a new position. Upon completion of the level, the tower moves to a new position.

A level “statue” consists of nine sublevels. As the user concentrates, the statue is gradually illuminated. After passing a sublevel, the user moves to a new statue. The statue level is the final level of the system 100 according to embodiments of the present invention.

TABLE 1

| | | Number | | | | Minimum |
|---|---|---|---|---|---|---|
| | | of | Positive | Negative | Reference | duration |
| Program | Level | sublevels | dynamic | dynamic | value | (Minutes) |
| Meditation | Forest | 5 | The statue is | Statue | 0.4 | 2.5 |
| Level 1 | | | reduced | grows | | |
| Meditation | Japanese | 7 | Stone appears | The stone | 0.6 | 2 |
| Level 2 | garden | | and grows | shrinks and | | |
| | | | | disappears. | | |
| Meditation | Island | 9 | The time | Reverse day | 0.8 | 2.5 |
| Level 3 | | | of day | shift (night, | | |
| | | | (morning, | evening, day, | | |
| | | | afternoon, | morning). | | |
| | | | evening, | | | |
| | | | night). The | | | |
| | | | morning is | | | |
| | | | different | | | |
| | | | from the | | | |
| | | | evening. | | | |
| Concentration | Targets | 5 | Archer pulls | Archer | 0.4 | 2.5 |
| Level 1 | | | the bow | releases | | |
| | | | string. | bowstring | | |
| | | | | bow. | | |
| Concentration | Towers | 7 | The ring of | The ring of | 0.6 | 2 |
| Level 2 | | | the tower | the tower | | |
| | | | dims at the | again appears | | |
| | | | old place and | on the old | | |
| | | | appears on | place and | | |
| | | | the new one. | disappears | | |
| | | | | on the new | | |
| Concentration | Statues | 9 | The | The | 0.8 | 2.5 |
| Level 3 | | | illumination | illumination | | |
| | | | of the statue | of | | |
| | | | rises. | the statue | | |
| | | | | decreases. | | |

The method 200 is further exemplified based on the level “Japanese garden”. A positive dynamics of the VR content 290 is the stone's size “growth,” while the negative dynamics of the VR content 270 is the stone's size “decreases,” which leads to its eventual disappearing. The reference value 250 of this level is 0.6. For example, upon entering the level a first actual meditation value for the user is 0.51. In this case, the VR content does not change (the stone remains the same size). After a second interval, the actual meditation value is 0.7. As the result, the edge of the stone appeared.

Another second later, the actual meditation value has increased to 0.72. As the result, the stone further increases in size slightly. After yet another measurement (in a second), the actual meditation value dropped to 0.01. As the result the stone decreased in size. A second later the actual meditation value becomes 0.64, and the stone increased in size again. This process continues until the stone reaches its maximum size (value). Upon reaching the maximum size of the stone, the user moves on to a second sublevel. In this case, the first stone remains in a given place in the user's field of vision, and a new stone appears next to the first stone. When the last stone reaches its final size, the “Japanese garden” level ends. If at some point during the exercise, the actual meditation value begin to fall steadily, the stone that is being “grown” in that instance by the user will decrease and disappear, but the previous stones will remain in place.

The method 200, as illustrated in FIG. 5, shows the steps of the meditation stage. The method 200 includes the meditation stage and the concentration stage. The steps of the concentrations stage are substantially the same as those described with respect to FIG. 5. While the method 200 includes both, the meditation stage and the concentration stage, either the meditation stage or the concentration stage can be used as a standalone process to achieve the results described herein by using the system 100.

The meditation stage of the method 200 can achieve in users a state of effective rest, stress relief (monitored, for example, by EEG and ECG). The concentration stage of the method 200 can achieve improved mental state and enhanced cognitive abilities than through other means of increasing concentration in a user. These results have been experimentally verified by administering tests relating to the concentration level to users before and after performance of method 200. With continuous use, a user can develop superior skills in managing his or her own psycho-physiological state, including superior ability to reduce stress and maintain cognitive alertness. These skills can be applied in a work environment or personal life. That is, the system 100 and method 200 disclosed herein can be used, for example, as an employment training tools to achieve higher cognitive concentration, improve productivity and as employees' stress release tool, classroom aid to elevate students' performance, and personal use to achieve higher levels of relaxation, stress release concentration and focus.

The foregoing detailed description of the embodiments is used to further clearly describe the features and spirit of the present invention. The foregoing description for each embodiment is not intended to limit the scope of the present invention. All kinds of modifications made to the foregoing embodiments and equivalent arrangements should fall within the protected scope of the present invention. Hence, the scope of the present invention should be explained most widely according to the claims described thereafter in connection with the detailed description, and should cover all the possible equivalent variations and equivalent arrangements.

The present invention can be a system, a method, and/or a computer program product. The computer program product can include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described herein with reference to flowchart illustrations and/or block diagrams of methods, apparatus (systems), and computer program products according to embodiments of the present invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the present invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form described. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A virtual reality (VR)-based interactive system for a neuro-meditation and neuro-concentration training, the system comprising:
a VR device having a display, the display is configured to provide a VR content;
a neural interface apparatus having a plurality of electrodes, each electrode is configured to receive a bio-signal data from a user;
a computing device having a processor,
wherein the computing device communicates with the neural interface apparatus and the VR device,
wherein the computing device continuously receives the bio-signal data from the electrodes, and
wherein the processor determines a feedback based on the bio-signal data and predetermined values and communicates the feedback to the VR device, the feedback being displayed as the VR content on the display of the VR device.

2. The system of claim 1 further comprising at least one hand controller for communicating with the VR device.

3. The system according to claim 1, wherein the neural interface apparatus comprises a positioning sensor.

4. The system according to claim 1, wherein the computing device is incorporated into the neural interface apparatus.

5. The system according to claim 1, wherein the computing device is incorporated into the VR device.

6. The system according to claim 5, wherein the predetermined values are a meditation coefficient and/or a concentration coefficient.

7. The system according to claim 6, wherein
the processor determines an actual meditation value and/or an actual concentration value based on the bio-signal data transmitted from the neural interface apparatus, the bio-signal data is determined based on the VR content,
the processor compares the actual meditation value and/or the actual concentration value with the meditation coefficient and/or the concentration coefficient to determine the feedback, the processor communicate the feedback to the VR device, and the VR device modifies the VR content in response to the feedback.

8. The system of claim 7, wherein the bio-signal data is determined every second.

9. The system of claim 7, wherein the VR device modifies the VR content positively or negatively based on the feedback.

10. A method for the neuro-meditation and the neuro-concentration training, the method comprising:
providing a VR device having a display, the display is configured to provide a VR content;
determining a bio-signal data of a user by a neural interface apparatus having a plurality of electrodes, each electrode is configured to receive the bio-signal data;
providing a computing device having a processor, wherein the computing device communicates with the neural interface apparatus and the VR device,
continuously receiving the bio-signal data from the electrodes by the computing device,
determining by the processor a feedback based on the bio-signal data and predetermined values;
communicating the feedback to the VR device; and
displaying the feedback as the VR content on the display of the VR device.

11. The method of claim 10, wherein the VR device further comprises at least one hand controller.

12. The method according to claim 10, wherein the neural interface apparatus comprises a positioning sensor.

13. The method according to claim 10, wherein the predetermined values are a meditation coefficient and/or a concentration coefficient.

14. The method according to claim 13 further comprising:
determining by the processor an actual meditation value and/or an actual concentration value based on the bio-signal data transmitted from the neural interface apparatus, the bio-signal data is determined based on the VR content,
comparing by the processor the actual meditation value and/or the actual concentration value with the meditation coefficient and/or the concentration coefficient to determine the feedback, communicating by the processor the feedback to the VR device, and modifying by the VR device the VR content in response to the feedback.

15. The method of claim 14, wherein the bio-signal data is determined every second.

16. The method of claim 15 further comprising modifying the VR content positively or negatively based on the feedback.

17. The method of claim 16 further comprising modifying the feedback positively if a feedback value is greater than the meditation coefficient and/or the concentration coefficient, as applicable.

18. The method of claim 16 further comprising modifying the feedback negatively if a feedback value is lower than the meditation coefficient and/or the concentration coefficient, as applicable.

19. The method of claim 10, wherein the method comprises a meditation stage and a concentration stage, and wherein the method further comprises performing the meditations stage before the concentration stage.

20. The method of claim 19, wherein each of the meditation stage and the concentration stage comprise levels and sublevels.

21. The method of claim 10, wherein the method comprises a meditation stage or a concentration stage.
